(19) United States
(12) Patent Application Publication
(10) Pub. No.: US 2016/0025729 A1
(43) Pub. Date: Jan. 28, 2016

(54) Systems And Methods For Early Detection Of Cervical Cancer By Multiplex Protein Biomarkers

(71) Applicant: OncoGenesis Inc., Morgan Hill, CA (US)

(72) Inventors: Nam Woo Kim, San Jose, CA (US); Peter Paul Gombrich, Salinas, CA (US)

(21) Appl. No.: 14/810,333

(22) Filed: Jul. 27, 2015

Related U.S. Application Data
(60) Provisional application No. 62/029,377, filed on Jul. 25, 2014.

(51) Int. Cl. G01N 33/574

(52) U.S. Cl. CPC G01N 33/57411; G01N 2333/8139; G01N 2333/025; G01N 2333/4742; G01N 2333/4704; G01N 2333/912; G01N 2333/9128; G01N 2333/705; G01N 2333/46

(57) ABSTRACT

Method for diagnosis and prognosis of premalignant and malignant cervical disease by using multiple neoplastic protein biomarkers are provided. In particular, methods and systems for screening cervical cells for the expression of proteins, which occur as a result of premalignant cervical disease and progression to invasive cervical cancer.

### CROSS REFERENCE TO RELATED APPLICATIONS

This application claims the benefit of, and priority to, U.S. Provisional Patent Application Ser. No. 62/029,377, filed Jul. 25, 2014, titled “EARLY DETECTION OF CERVICAL CANCER BY MULTIPLEX PROTEIN BIOMARKERS,” the entire disclosure of which is hereby incorporated by reference in its entirety.

### FIELD

Embodiments of the present disclosure relate to systems and methods for screening biological samples for markers associated with premalignant malignant lesions of cervical cancer, and the cancer progression. In particular, the present disclosure provides methods, devices and systems for screening cervical cells for the expression of proteins, which occur as a result of cervical neoplasia and progression to invasive cervical cancer.

### BACKGROUND

According to a recent report from the World Health Organization, cervical cancer is one of the most deadly cancers for women in many parts of the world. Cervical cancer screening is commonly based on cytological and colposcopic/histological analyses. The generally accepted cytological smear of the cervix (Papanicolaou test or Pap smear) has led to a reduction in the incidences of and mortalities caused by cervical cancer in developed countries.

The common Pap smear detects cellular abnormalities and thus the development of potentially pre-cancerous lesions. In a Pap smear, the collected cells are placed on a glass slide, stained and examined by a specially-trained and qualified cytotechnologist using a light microscope. In short, it is a subjective analysis with several known disadvantages, such as an increase in false-negatives and equivocal results as a consequence of debris obscuring abnormal cells.

The single most important risk factor for development of cervical cancer is human papillomavirus (HPV) infection. Although over 100 strains of HPV have been identified, only a subset is classified as high-risk (16, 18, 31, 33, 35, 39, 45, 51, 52, 56, 58, 59, 68, 73, and 82), or probable high-risk (26, 53, and 66) types (Munoz et al., NEJM, 348:518-527, 2003). Of these HPV types, HPV16 and HPV18 are reported to cause nearly 70% of all cervical cancer cases.

HPV-induced cervical cancer involves the following steps: (1) initial HPV infection, (2) persistent HPV infection, (3) transforming HPV infection, in the presence or absence of integration of HPV DNA into the host cell genome, (4) development of precancerous lesions and (5) development of invasive cancer. Evidence of HPV infection is very common, especially in young women. However, HPV infections typically resolve on their own or are suppressed by the immune system without causing serious pathology (e.g., advanced cervical disease including cervical intraepithelial neoplasia 2 (CIN 2), CIN 3 and invasive cancer). Thus, HPV infection alone does not detect or comment on the presence or the state of cervical lesions, and thus, makes the treatment decisions for patients with positive HPV test extremely difficult.

Cancer is a complex and multifaceted disease that requires multiple genetic and biochemical abnormalities at a cellular level. These abnormalities include growth deregulations, increased invasive properties, and prevention of programmed cell death. Thus, it is exceedingly complex to fully characterize key cellular changes during carcinogenesis.

On its own, a Pap smear permits identification of abnormal cervical cells through cell morphology, but not the disease status of the cells in molecular level. HPV tests permit the identification of HPV infection but not the presence of abnormal cervical cells. Thus neither test by itself is sufficient for detecting the presence of precancerous cells in a molecular level. Given these limitations, there is a pressing need for a single, non-morphological test that can address whether abnormal changes that could lead to cancer occurred to a host cell. Ideally this test would be in the form of a quick, disposable, point-of-care, molecular, cervical cancer screening system in a kit format. The disposability and point-of-care aspects would not necessitate a laboratory infrastructure and as such would permit the test to be utilized globally. The present disclosure satisfies these needs.

### SUMMARY

Embodiments of the present invention generally relate to methods, device, and kit for screening biological samples for markers associated with abnormal cervical lesions. In particular, the present disclosure provides methods, devices and systems for screening cervical cells for the expression of protein biomarkers that occur because of abnormal molecular changes that occurred in cervical cells that will eventually progress to invasive cervical cancer.

As described above, cancer is a complex and multifaceted disease that requires multiple genetic and biochemical abnormalities at a cellular level. These abnormalities include growth deregulations, increased invasive properties, and prevention of programmed cell death. The inventors have determined that the only practical method to fully characterize key cellular changes during carcinogenesis is to use multiple biomarkers that will measure all of the critical cellular changes in a clinical sample. Embodiments of the present invention describe and provide multiple protein biomarkers that are proven to be important in cervical cancer in a population of cells. Examination of biomarker expression in a population of cells versus individual cell provides several advantages. First, measuring multiple biomarkers in mixed population is more efficient, cost-effective, and simple than examining levels of multiple biomarkers in individual cells, which is a significant advantage when one is trying to develop most cost-effective tests for wide implementation especially in developing countries. Second, measurement of multiple biomarkers in mixed population containing both abnormal and normal cells could potentially provide more information than examining individual cells due to a phenomenon referred to as “field effect”. Since cancer is a dynamic disease where the interactions between the abnormal cancer or precancerous cells and the normal cells that are surrounding the abnormal cells play important role in disease progression, the expression of biomarkers in the adjacent non-malignant cell population can be as important as the expressions in the malignant cells. Thus, measuring the expression of the multiple biomarkers in a mix population of cells can provide more sensitive means of detecting abnormality, especially if those abnormalities are at early stage when it is difficult to tell the difference between normal and abnormal cells in the individual basis.

In one embodiment of a method for cervical cancer screening is provided, the method includes diagnosing presence of premalignant and/or malignant lesions of cervical cancer by detecting levels of at least two protein biomarkers by testing cellular proteins extracted from cervical cytology samples. The protein biomarkers include at least two markers from those listed in Table 1 and/or Table 2. The method of the embodiment is adapted to detect binding of the neoplastic biomarker-reactive reagents to neoplastic biomarkers, if the neoplastic biomarkers are present in a sample that includes cervical cells. In this embodiment, detection of elevated levels of the neoplastic biomarkers is indicative of the presence of premalignant or malignant cervical cancer cells in the sample.

In another embodiment the method includes determining the presence of premalignant or malignant cervical disease by evaluation of the presence or the levels of neoplastic biomarkers in the samples. Depending on the biomarker used, the levels of the biomarker will determine the state and the rate of disease progression from normal to premalignant to malignant cervical disease.

In yet another embodiment the method employs various protein detection technologies that include antibody-based assay, ELISA, western blotting, mass spectrometry, protein microarray, flow cytrometry, immunofluorescence, immunocytochemistry, and a multiplex detection assay. These technologies are able to detect the presence of the level of the neoplastic biomarkers. For optimal quantification of the protein biomarkers, the preferred embodiment is application of antibody-based ELISA assay or its similar modifications where both capture and detector antibodies are used to increase specificity of the signal and allows for accurate quantification of the proteins.

In yet another embodiment the method employs antibody-based protein detection technology based on multiplex assay system. Multiplex assay system is a method and device that is able to detect and measure levels of more than one analyte at the same time. Any suitable multiplex assay may be used, such as optical detection based on absorbance, luminescence, or fluorescence. In one preferred embodiment of the present invention, a multiplex assay utilizes detection of the biomarkers using a 3D Carbon sensor utilizing electrochemical detection.

### BRIEF DESCRIPTION OF THE FIGURES AND TABLES

FIG. 1A presents data on sensitivity and specificity of the neoplastic biomarkers in identifying premalignant cervical lesions in immunohistochemical analyses.

FIG. 1B illustrates measurement of HPV E6 protein in cervical cytology samples resulting in elevation of E6 protein expression in high-grade cervical cytology samples from histologically normal samples as reported by Yang.

FIG. 2 shows levels of Keratin 17 in high-grade cervical cytology samples as reported by Shroyer.

FIG. 3 depicts results from different protein extract methods according to embodiments of the present invention.

FIG. 4 shows detection of HPV E7 proteins from varying number of Hela cells according to various embodiments of the present invention.

FIG. 5 illustrates a comparative analysis of 40 ThinPrep samples lysed, and protein extracted according to two lysis buffers with different pH (e.g. pH 7 and pH 11) preparation of the present inventions.

FIGS. 6A and 6B depict results from evaluation of the neoplastic biomarkers in protein extracts from cervical cytology samples in two different studies, according to embodiments of the present invention.

FIG. 7 shows a standard curve and signals generated with a K17 ELISA, according to embodiments of the present invention.

FIG. 8 shows results of experiments in the expression of K17 in protein extraces from cervical cytology specimens performed on 16 remnant ThinPrep liquid cervical cytoloty samples using a K17 ELISA, according to embodiments of the present invention.

### DETAILED DESCRIPTION

The following description sets forth exemplary methods, parameters and the like. It should be recognized, however, that such description is not intended as a limitation on the scope of the present disclosure but is instead provided as a description of exemplary embodiments.

Embodiments of the present invention provide methods, devices and kits for the accurate diagnosis and prognosis of premalignant, and malignant, cervical disease including cervical cancer by molecular detection of multiple markers associated with cervical disease. The primary focus of the method is the detection of premalignant and malignant cervical disease by detecting the presence of neoplastic cervical cell changes resulting from deregulated cervical cell proteins and HPV infection. This combined approach to assessing the disease using multiple markers that arise at different stages of disease progression results in a test that has fewer false positives and fewer false negatives. This reduction in false positive and false negative results yields a test with significantly higher sensitivity and specificity, across all patient age groups. The screening tools of the present disclosure will allow far less time from the patient, the physician, and laboratory testing personnel.

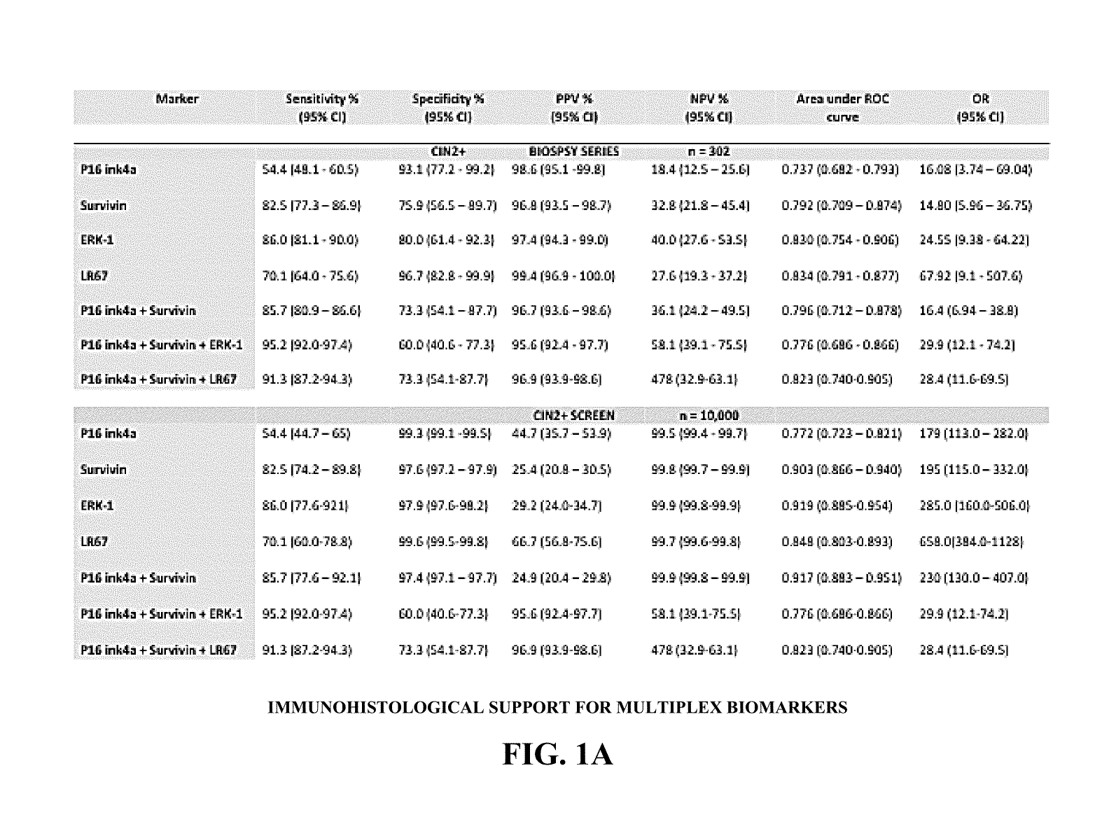

The cervical cancer screening method of the present disclosure comprises reagents for detection of at least two neoplastic markers (see FIG. 1 and Table 1 and 2). Detecting elevated expressions of neoplastic markers of cervical cancer in a single test, while optionally employing disease algorithms, increases the clinical relevance and confidence of the test. The neoplastic marker(s) are indicative of transcriptional or translational changes in the host cell that are associated with loss of cell cycle control and apoptotic processes leading to the development of cervical intraepithelial neoplasia (CIN) and ultimately to invasive cervical cancer.

In addition to the ability of the method to detect the presence of specific biomarkers, the inclusion of controls or standards within each test is critical for accurate determination of biomarker levels and changes as they relate to different stages of disease progression. Hence the method employs several controls independent of the specific mechanism for detection. Controls include the use of biomarkers for specific housekeeping proteins whose levels do not fluctuate with disease. Examples of such markers may include GAPDH, actin, B-globin, etc. Controls can be used to determine specificity of binding, noise, and/or be used to normalize the level of biomarker changes detected across numerous patient samples.

### Neoplastic Markers

With the advances in genomics and proteomics, a large number of cellular genes were found to be differentially expressed in cervical cancer cells in comparison to normal cervical epithelium (see, e.g., Santin et al., Virology, 331:269-291, 2005). Accordingly, a neoplastic cellular profile is assessed by detection of one or more neoplastic markers (e.g., reduced tumor suppressor levels or increased oncogene levels). In some preferred embodiments, the neoplastic marker(s) are host cell proteins that play roles in cell cycle progression or apoptosis. In exemplary embodiments, the neoplastic markers comprise p16INK4A, LR67, Erk-1, and survivin as shown in FIG. 1A and Table 1 and 2.

In HPV-associated tumors and dysplasia, the increased expression of HPV E7 results in down-regulation of Rb, hypomethylation of the p16INK4A promoter and marked overexpression of p16INK4A. Over-expression of p16INK4a represents a marker for CIN II and CIN III, as well as cervical carcinoma (Klaes et al., Int J Cancer, 92:276-284, 2001). It is also detected in the great majority of CIN I lesions associated with high risk HPV types, while no detectable expression of p16INK4A has been found in normal cervical epithelium or inflammatory lesions. p161NK4a (also referred to herein as p16 and p16^{INK4a}) is a cyclin dependent kinase inhibitor that plays a role in regulating cell cycle progression. It is expressed as isoform 1 along with several transcript variants from the CDKN2A gene. The amino acid sequence of p161NK4A is provided as GenBank Accession No. NP_{—}000068.

Elevation of E6 following HPV integration increases expression of survivin, via inhibition of p53 mediated transcriptional regulation. Analysis of survivin levels in cervical cancer samples show a strong correlation with high risk HPV and CIN grade. Therefore, both the survivin protein and mRNA are biomarkers for cervical cancer and its precursors (Branca et al., Amer J Clin Path, 12:113-121, 2005). Survivin (also referred to as apoptosis inhibitor 4, API4, baculoviral 1AP repeat-containing protein 5, and BIRC5) is a member of the inhibitor of apoptosis (1AP) gene family and as such plays a role in the control of programmed cell death. Several transcript variants of the BIRC5 gene have been identified, with the most frequently occurring transcript corresponding to isoform 1, encoding a protein with the amino acid sequence provided as GenBank Accession No. NP_{—}001159.

Other biomarkers that are upregulated or downregulated in cervical cancer cells are employed in further embodiments of the present screening tools. These markers may be involved in proliferation (Ki-67), associated with endocervical cells (Keratin 7), involved in cellular immortality (telomerase), or a stem cell marker (Cytokeratin 17).

In some embodiments, a multiplex biomarker platform is provided, comprising two or more neoplastic markers having properties or functionality as listed in Table 1 below:

TABLE 1
BIOMARKERS THAT DEFINES CANCER

| Biomarker | Details | Target(s) |
|---|---|---|
| Biomarker 1 | Tumor suppressor | Indicates uncontrolled growth |
| Biomarker 2 | Apoptosis inhibitor | Reflects prevention of cell death |
| Biomarker 3&4 | Viral oncoproteins | Indicate oncogenic viral |
| | | infection |
| Biomarker 5 | Proliferation Marker | Implicates abnormal growth |
| Biomarker 6 | Stem cell/reserve | Indicates more aggressive |
| | cell marker | tumors |
| Biomarker 7 | Housekeeping Protein | Normalization of cell sampling |

Given the teaching of the properties/functionality shown in Table 1 above, any suitable biomarker(s) may be used. In one exemplary embodiment, six biomarkers are used, comprised of: Biomarker 1: p16^{ink4a}, Biomarker 2: Survivin, Biomarker 3&4: HPV E6/E7, Biomarker 5: Ki-67, Biomarker 6: Keratin 17, and Biomarker 7: GAPDH.

In other embodiments, non-limiting examples of suitable neoplastic marker(s) are listed in Table 2 below (or otherwise mentioned above):

TABLE 2
LIST OF EXEMPLARY MULTIPLEX
NEOPLASTIC PROTEIN BIOMARKERS

| Biomarker | Details |
|---|---|
| p16^{ink4a} | Overexpressed in high risk cervical lesions in response |
| | to HPV E7 inhibition. |
| Survivin | Elevated in cervical lesions; inhibits apoptosis |
| HPV E6/7 | Viral oncoproteins elevated in cells infected with HPV |
| LR67 | Surface marker to enrich target traction |
| Keratin 17 | Stem cell/reserve cell marker |
| Keratin 7 | Associated with endocervical cells |
| Ki-67 | Involved in cell proliferation |
| Telomerase | Nuclear protein involved in cellular immortalization |

In one embodiment, the panel of biomarkers is comprised of biomarkers that are indicative of all critical molecular changes associated with cervical cancer progression. Application of these biomarkers in cervical cytology samples will provide a comprehensive assessment of the state of cervical cells from the patients and will be able to identify patients with high-grade lesions who require immediate follow-up or treatment accurately and efficiently. This test will be particularly useful for triaging HPV positive patients who truly need follow-up and/or treatment from the vast majority of HPV positive individuals who do not have abnormal lesions and who do not need expensive and stressful follow-up procedures.

Survivin is an inhibitor of the apoptosis protein family, and is expressed at high levels in malignant tumors (Fukuda S, Pelus L M (2006) Survivin, a cancer target with an emerging role in normal adult tissues. Mol Cancer Ther 5: 1087-1098). Specifically, HPV E6 gene up regulates survivin expression, which helps immortalize epithelial cells, leading to cell proliferation and tumor growth (Yaqin M, Runhua L, Fuxi Z (2007) Analyses of Bcl-2, Survivin, and CD44v6 expressions and human papillomavirus infection in cervical carcinomas. Scand J Infect Dis 39: 441-448. Borbély A A, Murvai M, Kónya J, Beck Z, Gergely L, et al. (2006) Effects of human papillomavirus type 16 oncoproteins on survivin gene expression. J Gen Virol 87: 287-294). Moreover, survivin has been shown to be an independent predictor of HSIL and above (Yaqin. Branca, M. et al. (2005) Survivin as a Marker of Cervical Intraepithelial Neoplasia and High-Risk Human Papillomavirus and a Predictor of Virus Clearance and Prognosis in Cervical Cancer American Journal for Clinical Pathology; 2005, 124:113-121. Frost M, Jarboe E A, Orlicky D, Gianani R, Thompson L C, et al. (2002) Immunohistochemical localization of survivin in benign cervical mucosa, cervical dysplasia, and invasive squamous cell carcinoma. American Journal of Clinical Pathology 117: 738-744). p16^{ink4a} is a negative regulator of cellular proliferation that influences the retinoblastoma (Rb)-controlled checkpoint of the cell cycle, and prevents the G1-S-phase transition, thus decelerating cell proliferation. However, in HPV-related tumors Rb is inactivated by viral protein E7, which leads to increased cell proliferation and an accumulation of intracellular p16^{ink4a} (Lakshmi, S., et al., p16ink4a is a Surrogate Marker for High-Risk and Malignant Cervical Lesions in the Presence of Human Papillomavirus. Pathobiol. 2009, 76:141-148.Klaes R, Friedrich T, Spitkovsky D, Ridder R, Rudy W, et al. (2001) Overexpression of p16(INK4A) as a specific marker for dysplastic and neoplastic epithelial cells of the cervix uteri. Int J Cancer 92: 276-284. Sherr C J (1996) Cancer cell cycles. Science 274: 1672-1677.

In a number of studies, overexpression of p16^{ink4a} has been detected in greater than 90% of HSIL and invasive cervical cancer biopsies and was generally absent in staining of normal tissue and low-grade intraepithelial lesions (LSIL) (Klaes R, Friedrich T, Spitkovsky D, Ridder R, Rudy W, et al. (2001) Overexpression of p16(INK4A) as a specific marker for dysplastic and neoplastic epithelial cells of the cervix uteri. Int J Cancer 92: 276-284. Sano T, Oyama T, Kashiwabara K, Fukuda T, Nakajima T (1998) Expression status of p16 protein is associated with human papillomavirus oncogenic potential in cervical and genital lesions. Am J Pathol 153: 1741-1748. Samarawardana P, Dehn D L, Singh M, Franquemont D, Thompson C, et al. (2010) p16(INK4a) is superior to high-risk human papillomavirus testing in cervical cytology for the prediction of underlying high-grade dysplasia. Cancer Cytopathol 118: 146-156. Nasreddine N, Borde C, Gozlan J, Bélec L, Maréchal V, et al. (2011) Advanced glycation end products inhibit both infection and transmission in trans of HIV-1 from monocyte-derived dendritic cells to autologous T cells. J Immunol 186: 5687-5695. Singh M, Mockler D, Akalin A, Burke S, Shroyer A L, et al. (2012) Immunocytochemical colocalization of P16(INK4a) and Ki-67 predicts CIN2/3 and AIS/adenocarcinoma. Cancer Cytopathol 120: 26-34). Furthermore, evaluation of p16^{ink4a} in cytology specimens using ELISA showed 91.8% sensitivity and 86.0% specificity for referral to colposcopy compared to conventional Pap test evaluations (Kurshumliu, F., et al. p16ink4a in Routine Practice as a Marker of Cervical Epithelial Neoplasia, Gyn. Oncol. 2009, 115:127-131). Gene transfer studies have identified HPV E6 and E7 genes as major viral oncogenes because their corresponding proteins (E6 and E7) have profound effects on the modulation of host cell cycle regulation leading to uncontrolled cell proliferation and tumor growth (Mantovani F, Banks L (2001) The human papillomavirus E6 protein and its contribution to malignant progression. Oncogene 20: 7874-7887. Riley R R, Duensing S, Brake T, Münger K, Lambert P F, et al. (2003) Dissection of human papillomavirus E6 and E7 function in transgenic mouse models of cervical carcinogenesis. Cancer Res 63: 4862-4871). Importantly, E6 and E7 proteins appear to be virus and disease-state specific markers of HPV-associated cervical dysplasia (Emens, L., Survivin' Cancer. Cancer Biol. & Therap. 2004, 3:180-183. Müller M, Viscidi R P, Sun Y, Guerrero E, Hill P M, et al. (1992) Antibodies to HPV-16 E6 and E7 proteins as markers for HPV-16-associated invasive cervical cancer. Virology 187: 508-514). Ki-67 is a well-established marker of cell proliferation that has been shown to be complementary to p16^{ink4a} (Keating J T, Cviko A, Riethdorf S, Riethdorf L, Quade B J, et al. (2001) Ki-67, cyclin E, and p16INK4 are complimentary surrogate biomarkers for human papilloma virus-related cervical neoplasia. Am J Surg Pathol 25: 884-891). Keratin 17 is a novel biomarker for cervical cancer recently reported by Dr. Ken Shroyer at Stoney Brook Medical Center using proteomic approach applied to laser-dissected cervical tissue samples (Escobar-Hoyos LF1, Yang J, Zhu J, Cavallo J A, Zhai H, Burke S, Koller A, Chen E I, Shroyer K R., Keratin 17 in premalignant and malignant squamous lesions of the cervix: proteomic discovery and immunohistochemical validation as a diagnostic and prognostic biomarker. Modern Pathology, 2013, 1-10).

Papillomaviruses are DNA viruses with a double-stranded, circular DNA genome containing a coding region for late (L) genes, a coding region for early (E) genes, and a non-coding upstream regulatory region with binding sites for the various transcription factors controlling expression of the early and late genes. Two separate open reading frames in the late gene coding region encode viral capsid proteins L1 and L2. Eight open reading frames in the early gene coding region, encode eight viral early proteins, designated E1, E2, E3, E4, E5, E6, E7, and E8. HPV can be found in cervical material in non-integrated forms (episomal), integrated forms or in mixed forms.

Increased expression of the E6 and E7 oncoproteins, due to integration of HPV DNA into the host genome or other mechanism of disrupting E2-mediated inhibition of E6 and E7 expression, induces chromosomal instability (Vinokurova et al., Cancer Research, 68:307-313, 2008). The E6 and E7 oncoproteins in tum bind to host cell proteins causing a dysregulation of cell cycle progression and proliferation (Ganguly and Parihar, J Biosci, 34:113-123, 2009). Specifically, E6 in association with host E6AP (associated protein), which has ubiquitin ligase activity, acts to ubiquinate the p53 tumor suppressor leading to its proteosomal degradation. Similarly, E7 binds to the retinoblastoma (Rb) tumor suppressor, freeing the transcription factor E2F to transactivate its targets. The E7 oncoprotein further destabilizes cell cycle control through its interaction with the cyclin-dependent kinase inhibitor protein, p21. HPV E6 and E7 oncoproteins are found to be continuously produced in transformed genital tissues. These interactions set the stage for controlling host cell proliferation and differentiation (i.e., transformation), a first step in the conversion of normal cells to pre-neoplastic cells and ultimately to the full expression of cancer malignancy.

Accordingly, HPV infection may be assessed by detection of one or both of the viral E6 and E7 oncoproteins. Amino acid sequences of exemplary HPV E6 proteins and E7 proteins are disclosed in FIGS. 4A and 4B, and FIG. 5, respectively, of U.S. Patent Application Publication No. 2009/0104597 to Gombrich and Golbus, herein incorporated by reference for the teaching of HPV protein sequences. In some embodiments, the reagents employed to detect the HPV marker(s) detect all HPV subtypes. In other embodiments, the reagents employed to detect the HPV marker(s) detect high risk types or only those high risk types most frequently associated with cervical cancer (e.g., HPV16, 18, 31, 33 and 45). In further embodiments, HPV presence may be confirmed by detection of additional viral markers alone (e.g., E4, E5, etc.), or in specific ratio to other viral proteins.

### Antibodies

In some embodiments, the HPV and neoplastic biomarkers are detected using marker-reactive polyclonal or monoclonal antibodies. In an exemplary embodiment, HPV markers are HPV E6 and E7, and the neoplastic markers are p16ink4a, surviving, and others that are listed in Table 2. HPV E6 and E7 are detected with antibodies cross reactive with viral antigens from multiple HPV strains. Antibodies may be purchased or licensed from a commercial source or produced in house for inclusion in the Cervical Screening platform. Exemplary anti-p16ink4a monoclonals include JC2, JC4, and JC6 (Dai et al., Gastroenterology, 119:929-942, 2000; Furth et al., Neoplasia, 8:429-436, 2006; Gump et al., Cancer Research, 61:3863-3868, 2001; and Nielsen et al., Laboratory Investigation, 79:1137-1143, 1999). The neoplastic markers such as surviving and others listed in Table 2 may be detected with a rabbit or mouse monoclonal or polyclonal antibody obtained from a commercial source such as AbCam (Cambridge, England) or with select hybridomal clones generated in-house or through commercial vendors. The present disclosure is not limited to the detection of these biomarkers or the use of the specific antibodies listed herein for this purpose.

### Patient Population

One of the benefits of the cervical disease screening method of the present disclosure is that their use is not accompanied by an age recommendation. Specifically, unlike the tests of some of the prior art, the disclosed cervical cancer screening tools are appropriate for use with women of all ages, including young women who are typically excluded from use of the currently approved HPV tests that do not distinguish transient HPV infection from transforming HPV infection. Women under 30 years of age are currently an underserved population because many of them have been exposed to HPV and thus are likely to be scored as a false positive on the currently approved HPV tests.

### Other Diseases

In further embodiments, the screening tools of the present disclosure provide reagents for detection of additional markers for the detection of other infectious diseases of the cervix. The device may be further multiplexed so that the detection of multiple types of cervical infectious diseases (and cervical disease progression) and/or sexually transmitted diseases occurs on a single cartridge using the same sample. Other diseases of interest include markers for the presence of Herpes Simplex virus, Chlamydia and Neisseria gonorrhoeae, among others.

### Sample Collection

In one embodiment, test samples come (1) directly from a swab or other collection device, such as CerMed's CerMap or iPap collectors, or (2) indirectly from a liquid transport/storage/processing medium. Alternatively the sample comes from an “at home” collection technique. The self-collection and self-test options open the way for women who do not or cannot have access to physicians. Expert care can be sought if warranted by the at home test.

### Sample Processing

Sample processing may include two stages: (1) initial processing after collection, hereafter referred to as preprocessing, and/or (2) processing in the device for target biomarker detection, including signal enhancement. Preprocessing may be performed prior to introducing the sample to the test device, or be performed in the test device. In an embodiment with preprocessing outside the device, the collected sample (e.g., collected using a standard cervical brush) is placed into a patient sample preprocessing (PSP) device. The device contains both fixed and solution-based means for sample processing including such things as filters for course and fine level filtration of cellular debris, mucous, as well as solutions containing reagents to lyse the cells for detection. In current embodiments, preprocessing steps will facilitate complete cervical cell lysis and recovery of the protein fraction. Solutions included in the sample processing device may include reagents that disrupt red-blood cells, remove cell clusters, degrade nucleic acids, inhibit protein degradation, alter membrane permeability promoting intracellular transport of antibodies to target proteins, and/or facilitate complete cervical cell disruption. Alternative approaches may also include addition of antibodies to surface markers indicative of target squamous epithelial cells, such as EpCam or Keratin proteins, to enrich specific cell fractions prior to analysis on the cartridge.

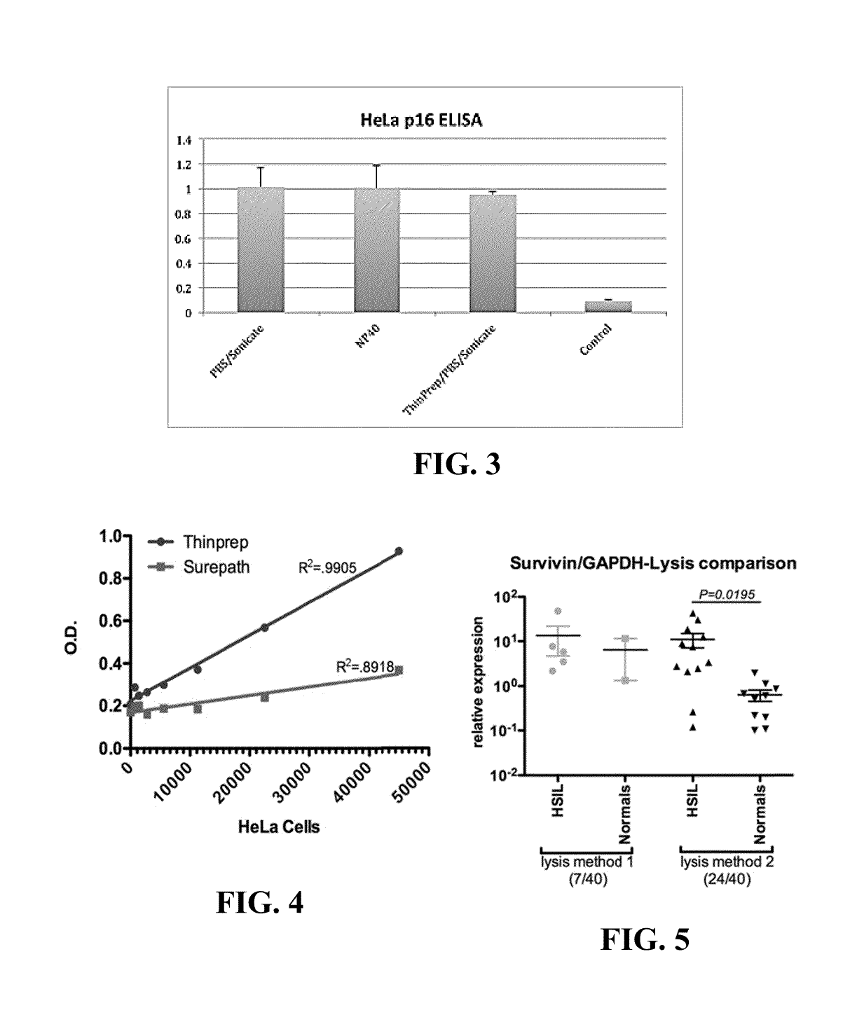

Common steps in sample preprocessing take place independent of the detection platform, however, specific steps will be required to facilitate neoplastic protein biomarker detection. In brief, cells are introduced into a device with a filter arrangement that filters large and small debris/contaminants and traps target cells. The device also contains reagents that disrupt clusters cells and promote cell lysis for subsequent detection of target markers. Lysis can occur as a final stage within the device or as an initial step on the device. Cell lysis can be achieved by a variety of common methods apparent to those familiar with the art. Lysis solutions may include addition of ionic or non-ionic detergents, protease, and/or phosphatase inhibitors, salts, buffers etc. hypotonic solutions, etc. A preferred cell lysis buffer includes a non-ionic detergent such as 1% Triton, or 1% NP-40 which is less denaturing to proteins than an ionic detergent, 20 mM Tris-HCL (pH 7.5), 150 mM NaCl, 1 mM Na2-EDTA, 1 mM EGTA, 1 mM B-glycerophosphate, 1 ug/mlleupeptin, 1 mM PMSF, and 1 mM benzamidine. An example of various methods for cellular protein extraction is presented in FIG. 3. In this experiment, we investigated the effect of different extraction methods for antigen retrieval and whether recovery of antigen from lysates prepared from cervical cells stored in cytological preservative (such as ThinPrep) was negatively impacted by the presence of mild fixatives used for cervical cell collection and storage. Results illustrating the relative absorbance (OD) FIG. 3 show that different recovery methods, including sonication or extraction with detergent such as NP40 provided similar levels for target p16 antigen, and that incubation of cultured cervical cells (HeLa) in ThinPrep preservative did not reduce amount of recoverable protein, further supporting our approach of detecting biomarkers in samples prepared in typical cytological preservative.

In an alternate approach, the target cell population can be enriched by employing antibody(s) against surface cell proteins specific for target cells. For CIN positive cervical squamous epithelial cells these markers may include but are not limited to molecules such as Ep-CAM, or specific isoforms of Keratin such as C5 or C14 (Litvinov, S. V. et al., 1996 Am J. Pathol 148(3); 865-875). Antibodies may be deposited within the sample device, presented on the surface of polystyrene or magnetic beads or upon a removable solid-state substrate with in the vial. A removable substrate can be subsequently transferred to an initial chamber within the device where captured cells are lysed and target antigens collected for subsequent detection. Alternatively, the initial chamber of the device contains an array or substrate displaying antibodies for target cell capture. Preprocessed cells are flowed over the cell capture substrate within the device, washed and lysed prior to collecting the lysate for detection.

Antibody coated beads or free antibodies presented in the sample processing device, or antibodies coated on available surfaces within the vial may also be used to directly complex target antigens during preprocessing steps as opposed to enriching for target cells. In this embodiment, cells are fully lysed within the sample processing device and the target antigens bound to free antibodies or antibodies complexed to polystyrene or magnetic beads or device surfaces. Target antigen-antibody complexes are washed within the device and the lysate collected for detection. Transfer may include the direct movement of antigen-antibody-bead complexes, or free antigen. Free antigen is attained by disruption of the antibody-antigen complex following washing in the device. Typical means for disruption include the use of buffers with increased salt concentration, detergents (SDS), etc. Preferred means include reversible processes such as employing buffers with increased salts, which can be removed by passing eluates through a subsequent desalting step or column.

In another embodiment target antigen enrichment in lysates can be completed prior to capture and analysis of proteins. Biomarkers listed on Table 2 exhibit relatively large molecular weights (>16 kDa). Therefore, methods that enrich for proteins of this size may improve sensitivity and reduce noise. Enrichment is achieved through any of a number of methods that reduce the fraction of proteins outside the molecular weight range of the target fraction. Protein concentration can be achieved through chromatographic means (size exclusion), filtration, or precipitation and re-suspension. In the preferred approach, lysates are processed via a size exclusion method, whereby specific fractions eluting from a chromatographic column or filtered on nanopourous membrane are collected and flowed over the capture array. Size exclusion may be implemented as a final step on a lysate flowing from the sample vial to the cartridge or as an early step on the cartridge prior to micro-electrode array capture. Typically, size exclusion chromatography employs columns with considerable length to improve separation of proteins over the course of travel. To accommodate dimensional constraints of the microfluidic cartridge, size exclusion resins or membranes may be employed within serpentine or linear paths within the cartridge, extending throughout one or more layers of the cartridge to increase the length of the flow path. Alternatively, a molecular weight cut-off filter may be employed to limit the flow of certain molecular weight fractions beyond a specific region of the cartridge.

Various resins, immunoaffinity monolith columns, or nanoporous microdialysis polymer membranes may also be employed within sample processing device for desalting or to filter/concentrate proteins of specific molecular weight. Use of these materials would aid in adjusting reaction conditions to promote increased binding between target antigens and antibodies used to enrich target cells or capture target proteins, to promote enzymatic activity critical for processing and/or detection or to isolate specific fractions of protein in a rapid manner.

An exemplary method for processing cells for analysis are as follows:
1. Spin down cytology samples (5-12 mL) in 15 mL Falcon at 1200 RPM for 10 minutes at room temperature (RT) and remove upper layer of fixative, leaving less than 1 mL of fixative/cells mixture.
2. Transfer all of mixture to 1.5 mL eppendorf tube and spin down for an addition 10 mins at 1200 RPM.
3. Discard supernatant.
4. Add 100 μL of Lysis Buffer consisting of 1% NP-40; 1× PBS; protease inhibitor (Halt Protease Inhibitor, Thermo Scientific) at pH 11, and mix using vortex for 30 seconds.
5. Incubate Lysis Buffer and cells for 30 minutes at room temperature and occasionally vortex solution. Store at 4° C.
6. Transfer 20 μL of cell extract to each microwell and proceed with ELISA protocol.

### Detection Method

Once the protein extracts are obtained from the cervical cytology samples, various protein detection technologies, that include antibody-based assay, ELISA, western blotting, mass spectrometry, protein microarray, flow cytrometry, immunofluorescence, immunocytochemistry, and a multiplex detection assay, can be used to detect the multiple neoplastic protein biomarkers. These technologies are able to detect the presence of the level of the neoplastic biomarkers. For optimal quantification of the protein biomarkers, the preferred embodiment is application of antibody-based ELISA assay where both capture and detector antibodies are used to increase specificity of the signal and allows for accurate quantification of the proteins.

An exemplary method for measuring a level of a neoplastic biomarker (Survivin) in cervical cytology protein extract is as follows:
1. Prepare Coating Solution: Add 10 μL of stock S-PC 1 capture pAb (1 mg/mL) into 10 mL PBS (Final concentration: 1 μg/ml). Mix well.
2. Coat plate with the Coating Solution, 100 μL/well, at 4° C., overnight.
3. Aspirate off coating buffer. No Wash.
4. Add 200 μL/well of SuperBlock PBS Blocking Solution (Pierce Cat #37515), cover and incubate at RT for 1 hour.
5. Discard blocking solution. Use the plate immediately, or let the plate dry inverted at RT for 2 hrs. Store the dried plate with desiccant at 4° C., and use the plate within one week.
6. In a separate, uncoated 96-well plate, add 20 μL of sample, or standard S-P1 (final concentration for top of standard curve is [500 ng/mL] followed by serial 3-fold dilutions; 500, 125, 31.25, 7.81, 1.953, 0.49, 0.12, 0 ng/mL). After serial dilutions, add 100 μL of Assay Buffer containing 10% FBS (HyClone Cat #SH30070)/1× PBS (Final top concentration after addition of assay buffer will be 500 ng/mL in 120 μL and so on for serial dilutions).
7. Add 100 uL of sample and standard to corresponding well in coated plate, and incubate for 1 hr with shaking @ 180 rpm at RT.
8. Wash plate 4× with 400 μl of 1× PBS-t (0.05%).
9. Prepare Dection Ab Solution: Add 4 μL stock S-MD1 detection mAb (1 mg/mL) into 10 mL assay buffer (10% FBS/1× PBS) (Final concentration: 0.4 μg/ml). Mix well.
10. Add S-MD1 detection Ab solution at 100 μL/well, and incubate plate with shaking @ 180 rpm, at RT for 1 hour.
11. Wash plate 4× with 400 μL of 1× PBS-t (0.05%).
12. Prepare Secondary Dection Ab Solution: Add 3.33 μL stock S-PS1 donkey anti-rabbit-HRP conjugated Ab (0.8 mg/mL) into 10 mL assay buffer (10% FBS/1× PBS) (Final concentration: 1:3000 dilution). Mix well.
13. Add 100 μL/well of S-PS1 Secondary Detection Ab solution, and incubate plate in dark with shaking @ 180 rpm, at RT for 30 min.
14. Wash plate 4× with 400 μl of 1× PBS-t (0.05%).
15. Add 100 μL of TMB substrate (Pierce Cat #34022) and incubate with shaking @ 180 rpm, at RT in dark for 20 min.
16. Add 100 μl/well of Stop Solution (VWR Cat #VW3202-1)
17. Read plate at 450 nm.

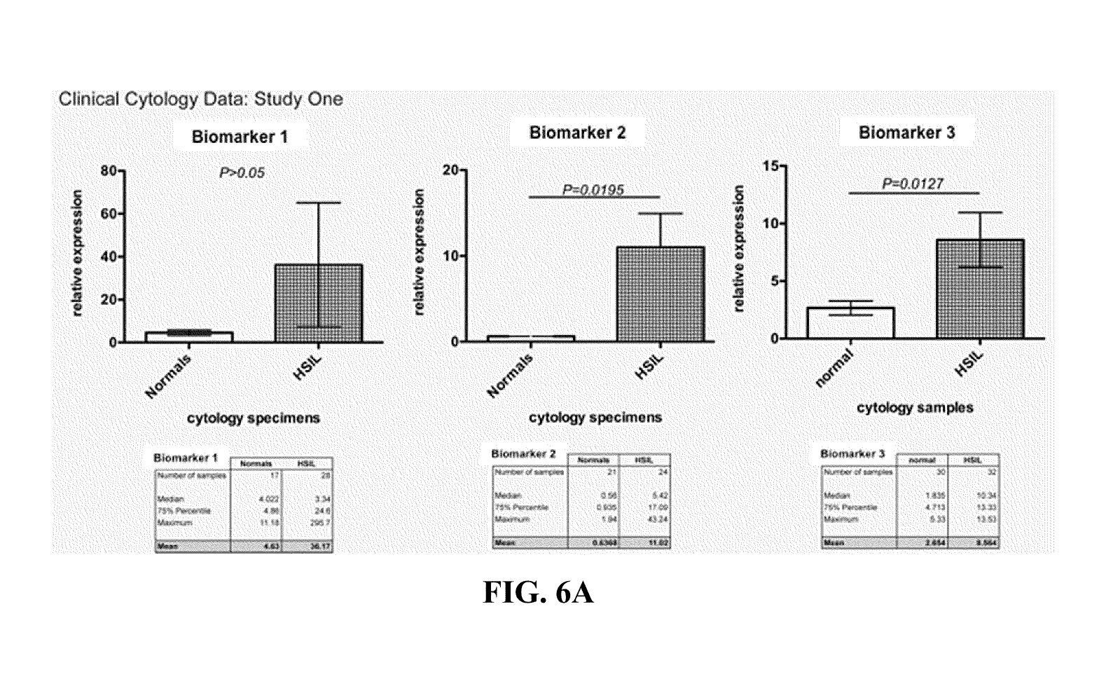

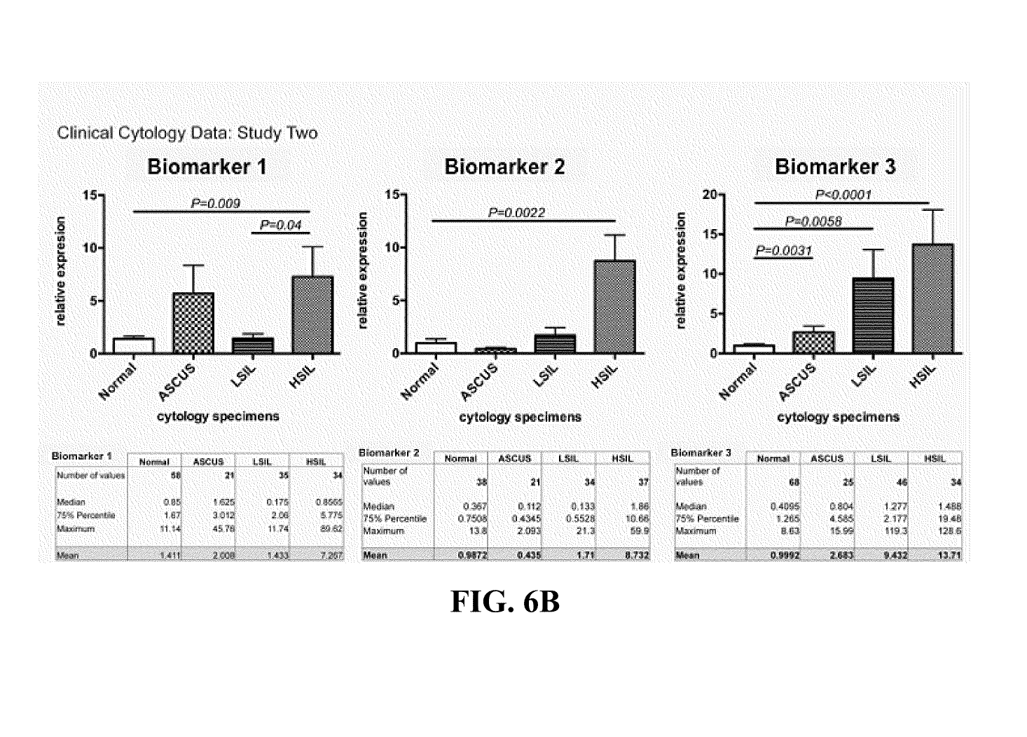

Using the sample preparation and ELISA immunoassay methods described above, we tested levels of 3 biomarkers (1: Survivin, 2: p16, 3: HPV E7) with GAPDH internal control in an initial set of 24 high-grade (HSIL) and 16 normal cervical cytology specimens (FIG. 6). The values of the biomarkers were then normalized to GAPDH internal control to account for the bias introduced by varying number of cells contained in each cytological specimen. The normalized values were then compared and analyzed using student t-test as shown below with mean and standard errors of the mean in FIGS. 6A and 6B. Importantly, these data show that the levels of all three biomarkers are elevated in high-grade cytology specimens when compared to normal controls. Even with limited number of samples, two of the three biomarkers achieved statistical significance in the separation of HSIL from normal controls.

In yet another embodiment the method employs antibody-based protein detection technology based on multiplex assay system. Multiplex assay system is a method and device that is able to detect and measure levels of more than one analyte at the same time. Any suitable multiplex assay system may be employed, such as but not limited to optical detection systems based on absorbance, luminescence, or fluorescence. In another embodiment the multiplex assay system is comprised of a 3D Carbon based sensor utilizing electrochemical detection to detect the biomarkers as described in more detail in U.S. Provisional Patent Application 62/036,040, filed Aug. 11, 2014, the entire disclosure of which is hereby incorporated by reference.

One advantage of the electrochemical biosensor over the typical optical detection method is that the electrochemical biosensor offers simpler measurements that can operate in turbid solutions that provide significant advantages in the analysis of biological samples. The signals from electrochemical can be detected in an electrochemical sensor by redox amplification, and since the amplified signal is purely electrical in nature, the potential background noise from biological samples is significantly reduced. Furthermore, the electrochemical sensor can be manufactured inexpensively using UV photolithography and can be miniaturized to fit any detection format. This mode of detection provides improved stability and robust functionality demanded of point of care systems while providing >10× the signal to noise ratio found with other carbon-based nanosensors.

In one embodiment, marker detection occurs in a disposable, point-of-care, cartridge or disc- based system housing incorporating carbon electrochemical sensor. This device allows the detection of up to 6 markers associated with the virus/disease at different states. The combination of markers allows a much higher level of sensitivity and specificity than available with one marker alone.

In one embodiment, the flow cytometric assay employs one or more MEMS components. In a preferred embodiment, the MEMS components comprise any required optical, actuator and manifold layers to optimize flow performance and, preferably, a minimum of 4-color detection in a fully contained, single, disposable point-of-care cartridge. Alternatively, the cartridge can be used in conjunction with a stand-alone reader capable of delivering any required reagents and processing signals originating from biosensors on the MEMS or non-optical, electrode based microfluidic cartridge. Both the MEMS and non-optical, electrode based approaches permit detection of multiple targets in a single specimen; the former in permeabilized whole cell preparations, the latter in cell lysates. Signal processing capability may, for example, reside with the MEMS, be contained within the reader or rely on components found both on the MEMS and the reader. The cartridge is also designed to facilitate any required preprocessing steps prior to target marker detection, and may employ filtration, exploit immunological detection of surface molecules on target cells, magnetic beads, etc., to ensure entry and flow of specimen to the MEMS chip.

In the preferred embodiment, a cartridge house multiple electrochemical-based 3-D Carbon sensors, comprise the array. Each sensor detects a specific analyte through catalytic reaction of electrochemical reaction dictated by specific biomarker probes. The test is designed to provide information to the user in a rapid, point-of-care manner, amenable to a low resource setting. This type of test could be performed in a lab. When the testing is performed in the lab, there is a physical and time separation from the patient and the answer. Thus, even if the results are negative, the patient still needs to return and re-enter the medical system. The 3D carbon based approaches described herein permit the test to be performed at the point-of-care and is especially applicable to developing countries lacking sophisticated medical and technical infrastructures. The separation of the patient, sample and test is minimal. The patient does not need to return for the results and appropriate utilization of the medical system is made possible.

To prepare the sample for subsequent analysis of the target markers, the preprocessing stage is designed to clean and concentrate target cells from unwanted material, such as blood or immune cells, cellular and non-cellular debris, digestion of mucous, and polysaccharide. Suitable tools for sample preprocessing may include, but are not limited to, membrane filtration, magnetic beads or other substrates displaying specific binding moieties for the physical and immunologically-based separation and concentration of target cells.

Membranes may include substrates modified with antibodies to collect target cells, or be designed based on size exclusion for filtering cells from extraneous material. Immunological enrichment steps may utilize general immunoglobulin-based capture or rely upon polystyrene or magnetic beads displaying specific reactive moieties to target cells. Preferred methods concentrate target cells or materials through capture on a surface or beads modified with antibodies or probes to cell surface or intracellular proteins, nucleic acid sequences, etc. Surface markers used to concentrate cells belong to classes of proteins that are commonly displayed on epithelial cells of the cervix (endo- and ectocervical region). Once attached to the enrichment substrate, target cells or material are washed free of unwanted components. As described above, steps may also include the use of resins or nanoporous polymer membranes to adjust buffer conditions or alter protein concentrations, promoting antigen-antibody binding.

Target cells are eluted from the preprocessing component (which may be on or off the cartridge/disc) and introduced into the processing stage of the cartridge. Once in the device there may be additional steps that further enhance the sample to prepare it for detection of target biomarkers. Sample processing may include washing of target cells and/or incubation with reagents that: lyse or permeabilize cellular membranes, promote antigen binding to antibodies for target cell surface proteins and/or intracellular markers, inhibit protein degradation, or utilize components for physical separation of target cells/proteins/nucleic acids from unwanted materials.

In a sample destined for analysis on a micro-electrode array cartridge, cells complexed to a substrate off-cartridge, such as magnetic beads, can be collected in an initial chamber within the cartridge. Conversely, preprocessed sample may be passed over magnetic beads displaying specific binding moieties for target cell capture, which are located within an initial chamber of the cartridge. Target cells are then washed and lysed in a reduced volume of mild, detergent-based lysis buffer (present as blister pack on the cartridge or added directly by the user) designed to disrupt cellular components freeing antigens for detection in subsequent stages. The lysate is then directed to a chamber housing the micro-electrode detection array via micro fluidic channels using manually or electronically controlled valves or pumps.

Detection on a 3D carbon electrode array consists of a substrate to which capture antibodies for target biomarkers have been bound. Lysate interacts/binds with antibodies to target markers and remaining material is washed free. Arrays may be multiplexed, containing all capture antibodies in defined locations, or be individually based, such that each array is designed for detection of a specific marker. The use of multiple arrays necessitates moving the lysate over each array or moving a portion of the lysate toward a specific array. Following binding to the detection array(s), the sample is washed on the array using wash buffer present on cartridge in blister pack or supplied separately. A second antibody, specific for each antigen is delivered to each array to form a sandwich. The second antibody optionally contains a component for signal amplification or signal detection. For example, signal amplification may be accomplished by addition of an antibody with a biotin or enzymatic conjugate, while signal detection may be facilitated by an antibody conjugated to a specific enzyme or fluorescent tag. Biotin conjugated antibodies support signal amplification through the use of secondary binding tools such as streptavidin-poly-horseradish peroxidase.

As mentioned above, cancer is a complex and multifaceted disease that requires multiple genetic and biochemical abnormalities at a cellular level, and these abnormalities include growth deregulations, increased invasive properties, and prevention of programmed cell death. One key aspect of embodiments of the present invention is the use of multiple biomarkers with a population of cervical cells, as opposed to a single cell. Of significant advantage, measurement of multiple biomarkers in mixed population containing both abnormal and normal cells could provide more information than examining individual cells due to a phenomenon referred to as “field effect” (Chai H, Brown R. E., Field Effect in Cancer-An Update. Annals Clinical and Laboratory Science, 2009, 39(4), 331-337).

Since cancer is a dynamic disease here the interactions between the abnormal cancer or precancerous cells and the normal cells that are surrounding the abnormal cells play important role in disease progression, the expression of biomarkers in the adjacent non-malignant cell population can be as important as the expressions in the malignant cells. Thus, measuring the expression of the multiple biomarkers in a mix population of cells could provide more sensitive means of detecting abnormality, especially if those abnormalities are at early stage when it is difficult to tell the difference between normal and abnormal cells in the individual basis.

Some initial work involved immunohistological and genomic testing of 302 biopsy samples against a panel of 13 different possible biomarkers has been reported (Branca, M. et al. Predicting High-Risk Human Papillomavirus Infection, Progression of Cervical Intraepithelial Neoplasia, and Prognosis of Cervical Cancer With a Panel of 13 Biomarkers Tested in Multivariate Modeling. International Journal of Gynecological Pathology. 2008, 27:265-273. Branca, M. et al. (2005) Survivin as a Marker of Cervical Intraepithelial Neoplasia and High-Risk Human Papillomavirus and a Predictor of Virus Clearance and Prognosis in Cervical Cancer American Journal for Clinical Pathology; 2005, 124:113-121); however no method or test has heretofore been reported as developed or enabled.

Experiments and study of the inventors has lead them to believe that a panel of 3 to 6 markers may be used to confidently grade cervical intraepithelial lesions. The inventors believe that high sensitivity and specificity for the detection of abnormal cervical lesions can be achieved by combining certain selected markers in multiplex fashion.

Multiple publications support the detection of human papillomavirus as an important metric for cervical cancer screening. In cytological settings this may often involve genomic screening of patient samples for evidence of HPV nucleic acids. Published evidence of the value of HPV in cervical screening, as well as evidence supporting its direct link to elevation in target cervical cell biomarkers has been identified Ressler, S., et al., High-Risk Human Papilloma virus E7 Oncoprotein Detection in Cervical Squamous Cell Carcinoma, Clin. Cancer Res. 2007, 13:7067-7072. Dehn, D., et al., Human Papillomavirus Testing and Molecular Markers of Cervical Dysplasia and Carcinoma, Cancer (Cancer Cytopathology) 2007, 111:1-14. Doorbar, J. Molecular Biology of Human Papilloma Virus infection and cervical cancer, Clin. Sci., 2006, 110:525-541. Moody, C. A., and Laimins, L. A. Human Papillomavirus Oncoproteins: Pathways to Transformation, Nature Rev., Cancer, 2010, 10:550-560). In a recent study by Yang et al. (Yang Y S1, Smith-McCune K, Darragh T M, Lai Y, Lin J H, Chang T C, Guo H Y, Kesler T, Carter A, Castle P E, Cheng S., Direct human papillomavirus E6 whole-cell enzyme-linked immunosorbent assay for objective measurement of E6 oncoproteins in cytology samples, Clin. Vaccine Immunol. 2012, 19(9):1474-1479) they showed that measurement of HPV E6 protein in cervical cytology samples resulted in elevation of E6 protein expression in high-grade cervical cytology samples from histologically normal samples, as shown in FIG. 1B.

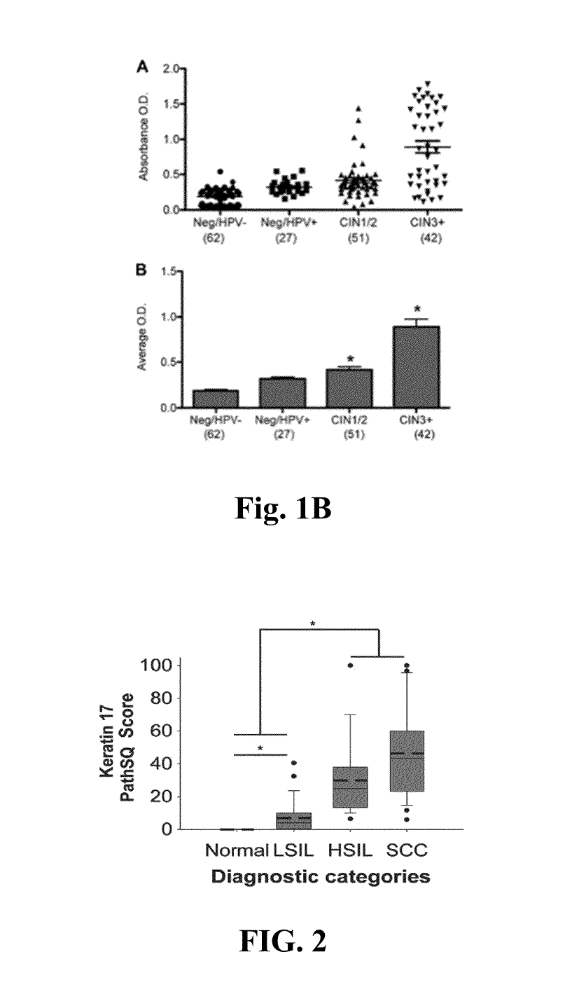

A recent manuscript by Dr. Ken Shroyer et al. at Stoneybrook Hospital describes novella potential biomarker for cervical cancer using proteomic approaches applied to laser-dissected cervical tissue samples (Escobar-Hoyos L F1, Yang J, Zhu J, Cavallo J A, Zhai H, Burke S, Koller A, Chen E I, Shroyer K R., Keratin 17 in premalignant and malignant squamous lesions of the cervix: proteomic discovery and immunohistochemical validation as a diagnostic and prognostic biomarker. Modern Pathology, 2013, 1-10). Specifically, it is reported that the level of Keratin 17 was significantly elevated in high-grade lesions and cervical cancer (FIG. 2), and was highly correlative with the progression of disease. While possible theoretical approaches have been reported, no method or test has heretofore been reported as developed or enabled.

### EXPERIMENTAL

A number of studies and experiments have been conducted by the inventors to show whether protein biomarkers in cervical cytology samples can provide clinically useful information in identifying high-grade cervical lesions. Results from several ELISA immunodetection assays developed according to embodiments of the present invention confirm the utility of biomarkers; demonstrating successful detection within both cultured cervical cell lines or cervical cytology specimens.

In an initial study, the effect of different extraction methods for antigen retrieval was investigated and whether recovery of antigen from lysates prepared from cervical cells stored in cytological preservative (such as ThinPrep PreservCyt) was negatively impacted by the presence of mild fixatives used for cervical cell collection and storage. Results (FIG. 3) illustrating the relative absorbance (OD) show that different recovery methods, including sonication or extraction with detergent provided similar levels for target antigens, and that incubation of cultured cervical cells (HeLa) in ThinPrep preservative did not reduce amount of recoverable protein, further supporting our approach of detecting biomarkers in samples prepared in typical cytological preservative.

To compare different liquid cytology samples in respect to compatibility with our test, we have evaluated protein extracts from samples stored in ThinPrep and SurePath preservative solutions with our biomarkers. FIG. 4 shows detection of HPV E7 proteins from varying number of Hela cells incubated overnight in ThinPrep and SurePath preservative solutions. Data clearly shows that the recovery of E7 antigen from ThinPrep is significantly higher than the SurePath preservative. Although both preservatives are alcohol-based, the inclusion of formaldehyde in the SurePath preservative is most likely the cause of poor protein recovery and/or antigenicity in ELISA. Thus, we have determined that the samples stored in ThinPrep, or other alcohol-based cytology preservative without formaldehyde, is a preferred method of choice for the test.

Since the samples stored in liquid cytology preservatives are mildly fixed and dehydrated by the presence of alcohol, efficient extraction of proteins from the liquid cytology samples is an important element in the method of the present invention. In order to determine the optimal extraction procedures for the liquid cytology samples, we have developed and evaluated different extraction procedures in liquid cytology samples. FIG. 5 shows a comparative analysis of 40 ThinPrep samples lysed, and protein extracted, using lysis buffer of two different pH (method 1: pH7, method 2: pH 11). The data shows that the levels of biomarker Survivin (normalized to GAPDH) were significantly different between the two methods, where method 2 resulted in significantly more detectable signals (24 of 40 samples detectable) than method 1 (7 of 40 samples detectable). Thus, the lyse/extraction method 2 was used to perform all subsequent biomarker analysis using the ThinPrep liquid cytology samples.

Using the optimal sample preparation method in ELISA immunoassays, we tested levels of 3 biomarkers with a cellular internal control in an initial set of more than 200 cervical cytology specimens, shown in FIGS. 6A and 6B. The two studies shown conducted using remnant cervical cytology specimens collected at local and national reference laboratories. The samples were stored at either 4° C. or room temperature for an average time of 43.4 days (range: 30-60) after collection by a health care professional. Samples were processed by a technique developed in our laboratory in which remnant preservative is removed and cells are concentrated prior to being lysed. This concentrated cell lysate was then used for quantification of specific cervical dysplasia biomarkers using our in-house immunoassay.

For the study, proteins were extracted from ThinPrep cervical cytology samples and the levels of 3 biomarkers and GAPDH internal control were measured using our ELISAs. The values of the biomarkers were then normalized to GAPDH internal control to account for the bias introduced by varying number of cells contained in each cytological specimen. The normalized values were then compared and analyzed using student t-test as shown below with mean and standard errors of the mean in FIGS. 6A and 6B. Importantly, these initial data show that the levels of all three biomarkers are elevated in high-grade cytology specimens when compared to normal controls. Even with limited number of samples, two of the three biomarkers achieved statistical significance in the separation of HSIL from normal controls.

These data are significant for at least the following reasons: The data show that using the optimized sample preparation/extraction method according to embodiments of the present invention the inventive multiplex biomarkers described herein can be reliably recovered from the standard cervical cytology samples for testing. The levels of biomarkers can differentiate samples from patients with high-grade cervical lesions from the normal controls. These positive results were obtained even though we were using “old” specimens due to sample availability (stored 3-6 weeks at room temperature prior to testing), indicating that the analytes are relatively stable in the ThinPrep liquid cytology preservative solution, and that the performance of our biomarkers could be increased with better quality samples. Performance of the biomarkers obtained from histological samples observed in the previous studies could be reproduced in cervical cytology samples.

Recent study has showed that the level of Keratin 17 was significantly elevated in high-grade lesions and cervical cancer (FIG. 2), and was highly correlative with the progression of disease (Escobar-Hoyos L F1, Yang J, Zhu J, Cavallo J A, Zhai H, Burke S, Koller A, Chen E I, Shroyer K R., Keratin 17 in premalignant and malignant squamous lesions of the cervix: proteomic discovery and immunohistochemical validation as a diagnostic and prognostic biomarker, Modern Pathology, 2013, 1-10.).

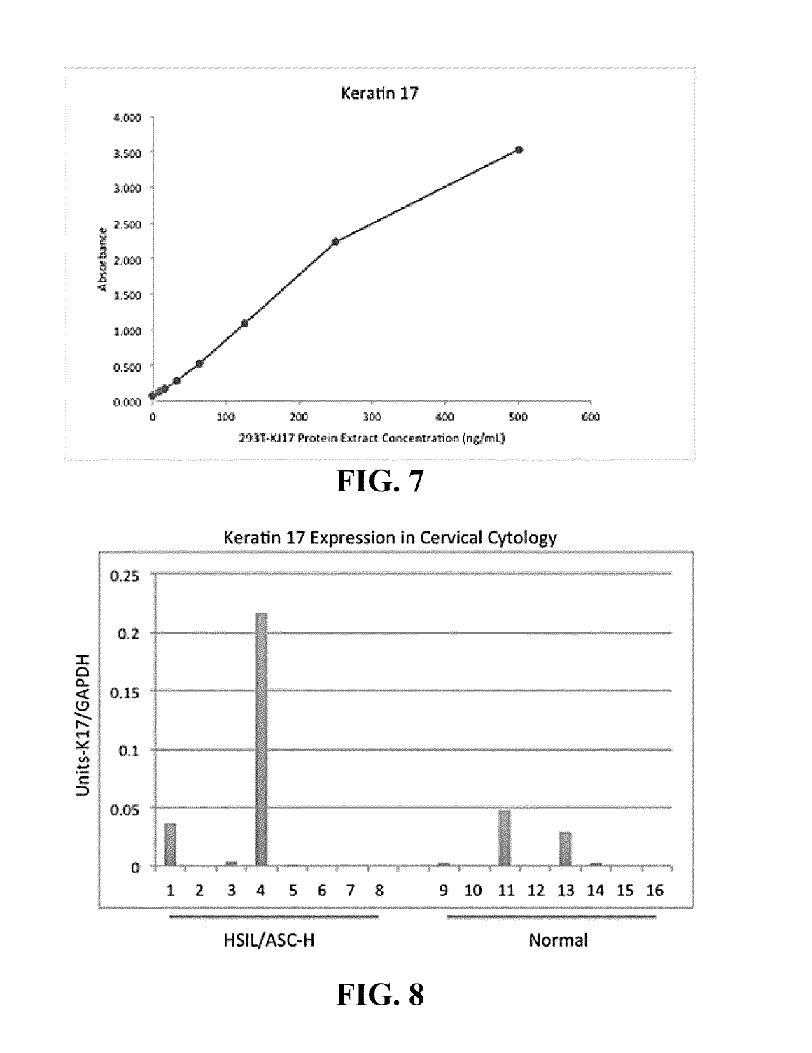

Since the original study examined the expression of Keratin 17 in tissue samples, we have examined the expression of Keratin 17 (K17) protein in cervical cytology samples. A sensitive ELISA for K17 using two commercially available antibodies from (AbCam, Cambridge, Mass.) that used a mouse monoclonal antibody (ab75123) to coat the ELISA plate and rabbit polyclonal antibody (ab53707) for detection has been developed. For calibration, a protein extract from 293T cells expressing K17 protein (ab94233) was used to quantify the signals. FIG. 7 and Table 3 below show the typical standard curve and signals generated with the K17 ELISA.

TABLE 3

| OD | | Mean OD | Std dev | % CV | ng/mL |
|---|---|---|---|---|---|
| 3.557 | 3.497 | 3.527 | 0.042 | 1.2 | 500 |
| 2.401 | 2.071 | 2.236 | 0.234 | 10.4 | 250 |
| 1.133 | 1.037 | 1.085 | 0.068 | 6.3 | 125 |
| 0.561 | 0.505 | 0.533 | 0.039 | 7.4 | 62.5 |
| 0.275 | 0.276 | 0.275 | 0.001 | 0.3 | 31.3 |
| 0.179 | 0.159 | 0.169 | 0.014 | 8.4 | 15.6 |
| 0.130 | 0.121 | 0.126 | 0.006 | 4.8 | 7.8 |
| 0.082 | 0.080 | 0.081 | 0.002 | 2.2 | 0 |

Expression of K17 in protein extracts from cervical cytology specimens were performed on 16 remnant ThinPrep liquid cervical cytology samples using the K17 ELISA. The samples consisted of 8 abnormal (HSIL or ASC-H) and 8 normal controls. The samples were extracted using our standard protocol (see attached), and the resulting protein extracts were tested using the K17 and GAPDH ELISAs. The K17 signals from the ELISA were then normalized to the corresponding GAPDH signals from each sample. The result of the study is presented in the FIG. 8.

The preliminary data shows detectable expression of K17 in 8 of the 16 samples. It was interesting to note that the highest signal came from abnormal (ASC-H) samples. However, the data needs to be confirmed in a larger study using higher quality samples. While it is unclear why many samples were not positive for K17, possible explanations include the degradation of the K17 protein in storage (samples were received 6-8 weeks after collection), insufficient number of cells in the samples (as noted by very low GAPDH levels), and some K17 proteins not being detected by our K17 ELISA.

The complete protocol for performing the ELISA described herein are prepared as follows:

### ELISA Protocol-Keratin 17

1. Prepare coating solution by making a 1:300 fold dilution of mAb (AbCam Cat #ab75123) into 1× PBS. Mix well.
2. Pipette 100 μL of coating solution into each well and incubate at 4° C. overnight.
3. Discard the coating solution. Do not wash.
4. Add 200 μL of SuperBlock PBS Blocking Solution (Pierce Cat #37515) to each well, cover with a plastic seal and incubate at room temperature (RT) for 1 hour.
5. Discard blocking solution. Use the plate immediately, or let the plate dry inverted at RT for 2 hrs. Store the dried plate with desiccant at 4° C. and use the plate within one week.
6. Prepare a 2-fold dilution of standards Keratin 17 (AbCam Cat #ab94233) in Assay Buffer (10% Bovine Calf Serum/1× PBS). The initial concentration of the standard curve should be 500 ng/mL. A 2-fold dilution should yield the following concentration: 500, 250, 125, 62.5, 31.3, 15.6,7.81, 0.00 (ng/mL).
7. Pipette 20 μL of standards (prepared from step 6) or samples into their corresponding well on the coated plate, then pipitte 100 μL of Assay Buffer into each well. Incubate the plate at RT, 180 rpm for 1 hr.
8. Wash plate 4× with 400 ul of 1× PBS-t (0.05%).
9. Prepare Detection Ab Solution (Rb pAb to Keratin 17, ab53707) with a final concentration of 0.5 μg/mL in Assay Buffer.
10. Add 100 uL/well of detection Ab solution (prepared in step 9), and incubate plate at RT, 180 rpm for 1 hr.
11. Wash plate 4× with 400 uL of 1× PBS-t (0.05%).
12. Prepare Secondary Dection Ab Solution (goat pAb to Rb IgG-HRP, AbCam Cat #ab97051) with a 1:5000 dilution in Assay Buffer. (final concentration should be 0.2 μg/mL).
13. Add 100 μL/well Secondary Detection Ab solution (prepared in step 12) and incubate at RT, 180 rpm for 30 min.
14. Wash plate 4× with 400 ul of 1× PBS-t (0.05%).
15. Add 100 μL/well of TMB substrate (Pierce Cat #34022) and incubate at RT in dark for 20 min. (Shaking can be omitted).
16. Add 100 ul/well of Stop Solution (VWR Cat #VW3202-1) after 20 min incubation.
17. Read plate at 450 nm.

The present invention is not to be limited in scope by the specific embodiments disclosed in the examples which are intended as illustrations of a few aspects of the invention and any embodiments which are functionally equivalent are within the scope of this invention. Indeed, various modifications of the invention in addition to those shown and described herein will become apparent to those skilled in the art and are intended to fall within the appended claims.

What is claimed is:

1. A method of diagnosing or providing a prognosis for premalignant and malignant cervical disease in an individual, the method comprising the steps of:
isolating proteins from cervical cytology samples;
detecting two or more neoplastic protein biomarkers associated with cervical cancer in a biological sample from an individual;
measuring the presence and/or the level of said biomarkers in the sample, and
using the presence and/or the levels of the biomarkers for diagnosing or a prognosis for premalignant and malignant cervical disease.

2. The method of claim 1, wherein the method comprises the detection of neoplastic biomarkers selected from any two or more of: p16ink4a, Survivin, HPV E6, HPV E7, LR67, Keratin 17, Keratin 7, Ki-67, ERK-1, or Telomerase.

3. The method of claims 1 or 2, wherein the method comprises detecting the level of at least two neoplastic biomarkers by a method selected from the group consisting of an antibody based assay, ELISA, western blotting, mass spectrometry, protein microarray, flow cytrometry, immunofluorescence, immunohistochemistry, and a multiplex detection assay.

4. The method of claim 3, wherein the level of at least one neoplastic biomarker is detected by antibody based assay.

5. The method of claim 3, wherein the level of at least one neoplastic biomarker is detected by mass spectroscopy.

6. The method of claim 3, wherein the level of at least one neoplastic biomarker is detected by a multiplex assay.

7. The method of claim 6, wherein said multiplex assay is antibody-based.

8. The method of claim 6, wherein said assay is electrochemical detection assay.

9. The method of claims 1 or 2, further comprising: determining whether or not two or more biomarkers are differentially expressed.

10. The method of claim 9, wherein the step of determining whether or not two or more biomarkers are differentially expressed comprises the steps of: (a) determining the level of said two or more neoplastic biomarkers in a sample from the individual; and (b) comparing said level to at least a first reference level from an individual not suffering from cervical disease.

11. The method of claims 1 or 2, wherein the cervical disease is squamous cell carcinoma (SCC).

12. The method of claims 1 or 2, wherein the cervical disease is High grade squamous intraepithelial lesion (HSIL).

13. A method of diagnosing or providing a prognosis for cervical cancer in an individual, the method comprising the step of detecting at least two neoplastic biomarker in a cytology sample from an individual, wherein said biomarker is selected from any two or more of: p16ink4a, Survivin, HPV E6, HPV E7, LR67, Keratin 17, Keratin 7, Ki-67, ERK-1 or Telomerase.

14. A method of diagnosing or providing a prognosis for cervical cancer in an individual, the method comprising the steps of:
contacting a cervical biological sample from said individual with a reagent that specifically binds to two or more neoplastic biomarkers; and
determining in a multiplex assay whether or not said more than one neoplastic biomarkers are differentially expressed in the sample, thereby diagnosing or providing a prognosis for cervical cancer, wherein said two or more protein biomarkers are selected from any two or more of: p16ink4a, Survivin, HPV E6, HPV E7, LR67, Keratin 17, Keratin 7, Ki-67, ERK-1, or Telomerase.

15. The method of claim 14, wherein said multiplex assay is antibody based.

16. The method of claim 14, wherein said multiplex assay is electrochemical based.

17. The method of claim 14, wherein said multiplex assay is protein microarray.

18. A method of diagnosing or providing a prognosis for either HSIL or cervical cancer in an individual, the method comprising the steps of: (a) contacting a cervical biological sample from an individual with a reagent that specifically binds to two or more cervical cancer biomarkers; (b) determining in a multiplex assay the level of expression of said two or more cervical cancer biomarkers; and (c) classifying the level of expression as either a first, second, or third level; wherein, said first level corresponds to a diagnosis of no HSIL or cervical cancer, said second level corresponds to a diagnosis of HSIL, and said third level corresponds to a diagnosis of cervical cancer.

19. The method of claim 18, wherein the step of classifying the level of expression comprises comparing the expression profile of said neoplastic biomarkers to at least a first reference expression profile.

20. A method of diagnosing or providing a prognosis for HSIL in an individual, the method comprising the steps of: (a) detecting two or more neoplastic biomarker in a biological sample from an individual; and (b) determining whether or not said biomarkers are differentially expressed in the sample, thereby diagnosing or providing a prognosis for HSIL.

21. The method of claim 20, wherein determining the expression level of said at least two biomarkers comprise performing a multiplex antibody-based assay.

22. A kit for use in diagnosing or providing a prognosis for cervical cancer in an individual, the kit comprising at least two reagents that specifically binds to two neoplastic biomarkers.

23. The kit of claim 22, wherein said reagents are multiplex reagents capable of binding to two or more neoplastic protein biomarkers.

24. The kit of claims 22 or 23, wherein at least two neoplastic biomarkers are selected from any two or more of: p16ink4a, Survivin, HPV E6, HPV E7, LR67, Keratin 17, Keratin 7, Ki-67, ERK-1 or Telomerase.

25. The method of claim 1, wherein the cervical cytology samples are from HPV-positive patients.
